(19) United States
(12) Patent Application Publication
(10) Pub. No.: US 2025/0120669 A1
(43) Pub. Date: Apr. 17, 2025

(54) METHOD AND APPARATUS FOR COVERING A STETHOSCOPE

(71) Applicant: Cynthia Anne Valdez, Los Ranchos, NM (US)

(72) Inventor: Cynthia Anne Valdez, Los Ranchos, NM (US)

(21) Appl. No.: 18/919,227

(22) Filed: Oct. 17, 2024

Related U.S. Application Data
(60) Provisional application No. 63/544,518, filed on Oct. 17, 2023.

(51) Int. Cl. A61B 7/02

(52) U.S. Cl. CPC A61B 7/02

(57) ABSTRACT

A stethoscope cover that can include a head, a neck extending from the head to an opening, and a rip cord that is imbedded or otherwise formed or disposed within and which extends along a length of the stethoscope cover to facilitate splitting the cover open for easy removal. A method of preserving sterility in a stethoscope that can include inserting a stethoscope into a stethoscope cover, performing tasks with the stethoscope cover over the stethoscope, pulling the rip cord, splitting the stethoscope cover, removing the stethoscope, and disposing of the stethoscope cover.

### CROSS-REFERENCE TO RELATED APPLICATIONS

This application claims priority to and the benefit of the filing of U.S. Provisional Patent Application No. 63/544,518, entitled “Method and Apparatus for Covering a Stethoscope”, filed on Oct. 17, 2023, and the specification thereof is incorporated herein by reference.

### BACKGROUND OF THE INVENTION

Embodiments of the present invention relate to a method and apparatus for covering a portion of a stethoscope. More particularly a method and apparatus for providing a sterile disposable stethoscope covering that can easily be removed and replaced to provide a clean and/or sterile stethoscope covering for use on each patient.

In 1985, the Center for Disease Control (“CDC”) developed universal precautions in response to the human immunodeficiency virus and acquired immunodeficiency syndrome (“HIV/AIDS”) epidemic. Personal protective equipment (“PPE”) was developed to protect healthcare workers (“HCW”). In 1996, standard precautions were recommended to be used with all patients with specific infections such as Clostridioides difficile (“C. difficile”). The term “nosocomial infection” was used for infections acquired in healthcare facilities. In 2006, guidelines to prevent nosocomial infections and multi-drug-resistant organisms (“MDRO”) were published.

Transmission of an infection requires 1) an infectious agent 2) a susceptible host with a portal of entry and 3) a mode of transmission of the infection. Most healthcare associated infections are transmitted from the patient, but can be transmitted by HCW's, and inanimate environmental sources, which can include for example healthcare equipment. Thus, it is now vitally important to protect each patient from cross contamination. Host susceptibility—for example extremes in age, diabetes, HIV/AIDS, malignancy, transplants, and medications that alter gut flora or the host's immune system can cause disease and/or sepsis from exposure to these pathogens. The most common mode of transmission is direct contact between a patient and HCW. Indirect transfer can also occur through a contaminated instrument or person, especially contaminated hands of HCW's. Patient care devices, including stethoscopes can transmit infection if not cleaned between patients. There are multiple organisms that are especially epidemiologically important, including C. difficile, prions, severe acute respiratory syndrome coronavirus (“SARS-COV”), monkey pox, norovirus and hemorrhagic fever viruses, Methicillin-resistant Staphylococcus aureus (“MRSA”), herpes simplex virus (“HSV”), Varicella, multidrug-resistant organism (“MDRO”), Vancomycin-resistant Enterococci (“VRE”), etc. These infections have a propensity for transmission in a healthcare facility, causing severe illness and anti-microbial resistance.

It is estimated that 1.8-13.5 million hospital associated infections are brought into nursing homes, correctional facilities and even home healthcare settings. Despite efforts to control infection, observational studies showed that only 27.8% of nurses and nurse assistants adhere to PPE, versus 100% of Infectious Disease Doctors. Antimicrobial resistance has become a global healthcare challenge. Previous studies estimate that 569,000 deaths were associated with antibiotic resistance.

Studies show that stethoscopes are often contaminated by pathogens and can harbor bacteria, and only rarely do HCW's clean their stethoscopes after use. Healthcare facilities have attempted to eliminate this source of cross contamination by providing disposable stethoscopes that are left at the patient's bedside and only used for that patient, but disposable stethoscopes stay in the environment of a patient with a nosocomial infection and therefore are often still contaminated. Existing disposable stethoscopes are typically not cleaned between use or replaced when contaminated. Existing disposable stethoscopes are expensive, of poor quality and very uncomfortable to use, making it very difficult for auscultation, rendering them almost useless. HCW's are often reluctant to use disposable stethoscopes. Most providers prefer their high-quality stethoscopes for good auscultation of heart, lung and abdominal sounds. As a result, stethoscopes remain a vector of transferring nosocomial infections from patient to patient.

Nosocomial infections are a major cause of patient morbidity and mortality and dramatically increase the cost of health care delivery. Just as health care delivery was made safer by introducing handwashing between patient visits in the 1800's, hospitals are now redesigning patient rooms, making them private to minimize the spread of nosocomial infections. There is a present need for a method and apparatus to sterilize stethoscopes between consecutive patient visits to aid in minimizing the risk of cross contamination between patients

Current procedures require a user to preferably wipe down the usable end portion of their stethoscope between patient visits. However, this practice is often not observed. And even when observed, it is possible for the stethoscope to become contaminated between patient visits. Still further, a “throwaway” stethoscope, when provided, is typically put in a room when a patient has been infected with an organism that is resistant to multiple antibiotics (for example methicillin-resistant Staphylococcus aureus (“MRSA”) or Clostridioides difficile). Unfortunately, these stethoscopes become contaminated by being in the room, on the patient, or on the floor at times. The user, therefore, is likely to carry drug-resistant organisms from the patient to another patient, to others in the healthcare facility, or to their families. There is therefore a present need for a disposable stethoscope cover in order to prevent the spread of resistant organisms.

Traditional stethoscope covers currently on the market only attach to the diaphragm. Traditional diaphragm covers are very small and prevent infection of only the surface of the bell, and therefore cannot effectively prevent transmission of pathogens. A stethoscope cover that is very long is difficult to use and can become contaminated when removing. A solution is needed to overcome these obstacles.

What is needed is a covering that eliminates the spread of pathogens via stethoscopes-particularly a covering that covers the lower third of the stethoscope, including both the bell and diaphragm. It needs to be easy to put on and pull off the stethoscope between each patient use without affecting the auditory qualities of the stethoscope, and which is inexpensive, can be thrown away after use and a dispenser box can be conveniently placed at every patient's door.

### BRIEF SUMMARY OF EMBODIMENTS OF THE PRESENT INVENTION

Embodiments of the present invention relate to a stethoscope cover having an elongated neck portion, the elongated neck portion having an opening at a proximal end thereof, a head disposed at a terminal end portion of the elongated neck portion, the head configured to accommodate a chest piece of the stethoscope, and a rip cord disposed within or positioned inside of the elongated neck portion. The stethoscope cover can also include a tension band disposed at or near a terminal end portion of the elongated neck portion, which can define a proximal end of the head. The tension band can delineate a transition between the head and the elongated neck portion. The tension band can include a thickness that is greater than a thickness of the head. The rip cord can extend at least from the opening to the head. The rip cord can extend at least from the opening and terminate within the head. The head can be a head portion of the stethoscope cover. The head can comprise a diameter which is larger than a diameter of the neck portion and/or can comprise a cross sectional area which is larger than a cross sectional area of a portion of the neck portion. Optionally, the rip cord can include a pull tab extending beyond the opening of the elongated neck portion.

In one embodiment, the rip cord can be embedded within material that at least forms the elongated neck portion. Optionally, the rip cord can be adhered to an internal surface of at least the elongated neck portion. The rip cord can include a pull tab extending from the head of the stethoscope. In one embodiment, the head comprises a flat portion that is positioned to accommodate a diaphragm of a chest piece of a stethoscope when a stethoscope is disposed within the stethoscope cover and wherein the head comprises dimensions that accommodate a chest piece of the stethoscope. The rip cord can be positioned opposite the flat portion—for example if a stethoscope chest piece is disposed within the head with the diaphragm against the flat portion, the rip cord can be disposed on or in the portion of the cover that contacts the bell of the stethoscope. The stethoscope cover can include a lip formed around the opening. The proximal end of the elongated neck portion can be flared such that the opening has a diameter that is larger than a diameter of a rest of the elongated neck portion.

Embodiments of the present invention also relate to a method of preserving sterility of a stethoscope, the method including disposing a stethoscope at least partially within a cover such that a chest piece of the stethoscope is positioned within a head of the cover and such that at least a portion of tubing of the stethoscope is disposed within a neck portion of the cover; pressing the head of the cover, having the chest piece disposed therein, against a patient to perform an auscultation; after the auscultation, pulling a rip cord such that the rip cord tears or otherwise cuts through at least a portion of the cover to facilitate removal of at least a portion of the stethoscope from the cover; and removing the cover from the stethoscope. Optionally, pulling a rip cord can include a user grasping a pull tab and pulling the pull tab to pull the rip cord. The method can also include disposing the cover after the removing step. In one embodiment, after disposing of the cover, the method can include at least partially inserting the stethoscope within a new cover. Pulling a rip cord can include pulling the rip cord from a top opening of the cover toward the head and/or pulling the rip cord from the head toward a top opening of the cover.

Objects, advantages and novel features, and further scope of applicability of the present invention will be set forth in part in the detailed description to follow, taken in conjunction with the accompanying drawings, and in part will become apparent to those skilled in the art upon examination of the following, or may be learned by practice of the invention. The objects and advantages of the invention may be realized and attained by means of the instrumentalities and combinations particularly pointed out in the appended claims.

### BRIEF DESCRIPTION OF THE SEVERAL VIEWS OF THE DRAWINGS

The accompanying drawings, which are incorporated into and form a part of the specification, illustrate one or more embodiments of the present invention and, together with the description, serve to explain the principles of the invention. The drawings are only for the purpose of illustrating one or more embodiments of the invention and are not to be construed as limiting the invention. Although the drawings illustrate an embodiment wherein the cover is formed from a transparent material in order to better illustrate a most preferred fitment, the material need not be transparent in order to provide desirable results. In the drawings:

FIG. 1 is a drawing which illustrates a top view of a stethoscope cover disposed on a stethoscope, according to an embodiment of the present invention;

FIG. 2 is a drawing which illustrates a side view of a stethoscope cover disposed on a stethoscope, according to an embodiment of the present invention;

FIG. 3A is a drawing which illustrates a top view of an end portion of a stethoscope disposed on a stethoscope according to an embodiment of the present invention;

FIG. 3B is a drawing which illustrates a side view of a stethoscope cover disposed on a stethoscope according to an embodiment of the present invention;

FIG. 4A is a drawing which illustrates a top view of an end portion of a stethoscope cover wherein a pull tab of the rip cord is exposed at a chest piece end of the stethoscope when the cover is installed on a stethoscope embodiment of the present invention;

FIG. 4B is a side view of the embodiment of FIG. 4A; and

FIG. 5 is a flow diagram showing a method of preserving sterility in use of a stethoscope, according to an embodiment of the present invention.

### DETAILED DESCRIPTION OF THE INVENTION

Embodiments of the present invention are a faster and easier method and apparatus to provide a stethoscope with a sterilized covering for use on a patient. Embodiments of the present invention provide a solution for HCWs in an emergency and can be used on all patients, even when a serious infection is not known to exist with the patient. As more resistant organisms are on the horizon, the present invention is needed to decrease the spread of these infections.

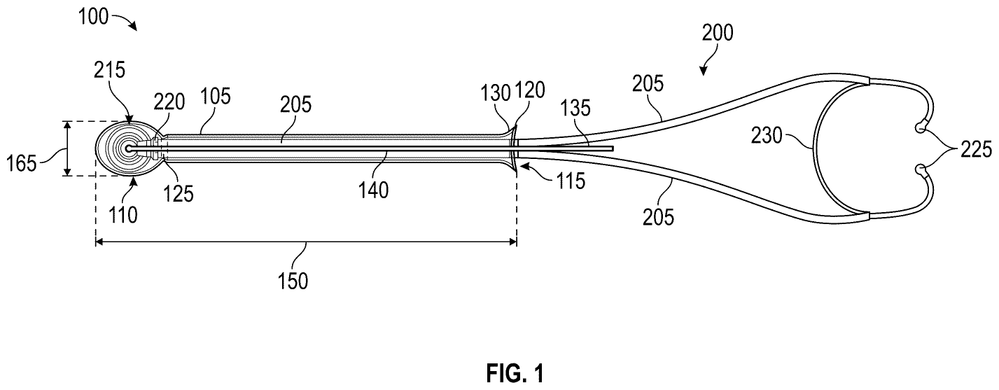

Referring now to the figures, FIG. 1 illustrates stethoscope cover 100, having neck 105, head 110, opening 115, and lip 120. Stethoscope 100 can be pulled over and around a portion of stethoscope 200. A typical stethoscope 200 can include one or more tubes 205, diaphragm 210, chest piece 215, stem 220, earpiece 225, and spring 230. Chest piece 215 is preferably inserted through opening 115 of stethoscope cover 100. A user can pull lip 120 to expand opening 115 and make it easier for chest piece 215 to be inserted. Neck 105 preferably tapers outwards to lip 120 at flared end 130. Flared end 130 is thus preferably wider than neck 105 and allows diaphragm 210 and the rest of chest piece 215 to be inserted into opening 115 and easily maneuvered from lip 120 into neck 105. In one embodiment, chest piece 215 is maneuvered down and through the length of neck 105 until chest piece 215 rests within head 110 of stethoscope cover 100. Head 110 is preferably circular or oval in shape to house chest piece 215 and to accommodate diaphragm 210. Tubes 205 rests within neck 105. Optionally, however, stethoscope cover 100 can be rolled inside our (for example by rolling lip 120 down until stethoscope cover 100 comprises a mostly flat, disc-like shape). Stethoscope cover 100 can thus be packaged in this flattened configuration such that when a user desires to install it onto stethoscope 200, a user can simply place an end portion of chest piece 215 into contact with stethoscope cover 100 and can then unroll stethoscope cover 100 so that lip 120 travels up along chest piece 215, then along tubes 205 toward ear piece 225 until stethoscope cover 100 is fully unrolled and at least a portion of stethoscope 200 is disposed therein.

Stethoscope cover 100 preferably also includes tension band 125. Tension band 125 can comprise a thickened portion of material between head 110 and neck 105. Because stethoscope cover 100 is preferably formed from an elastomeric material, the thickened portion that forms tension band 125 provides a more firm constriction ring to help keep chest piece 215 retained within head 110 of stethoscope cover 100. Tension band 125 is positioned just above head 110, within which chest piece 215 rests. Although Tension band 125 is preferably formed from a thickened ring of material from which stethoscope cover 100 is formed, tension band 125 can optionally be formed from an elastomeric material that is attached to an inside of stethoscope cover 100, an outside of stethoscope cover 100 or otherwise embedded within the material that stethoscope cover 100 is made of. In these cases, tension band 125 is preferably formed from an elastomeric material.

Stethoscope cover 100 further includes rip cord 140. An exposed end portion of rip cord 140 preferably extends outside of the rest of stethoscope cover 100 and provides an exposed material that can be grasped and pulled on by a user. In one embodiment, pull tab 135 extends outwards from or near lip 120. Pull tab 135 is connected to rip cord 140 and can optionally be nothing more than an end portion of rip cord 140. Pull tab 135 can comprise any desired shape or size to provide a portion that can be easily grasped by a user. Rip cord 140 extends from the center of head 110, along the length of neck 105, up flared end 130 and to pull tab 135. Rip cord 140 is preferably embedded within the material that stethoscope cover 100 is made of or is otherwise attached to an inside wall of stethoscope covering 100—for example via an adhesive. A user can grasp and pull on pull tab 135, which subsequently pulls rip cord 140 down along the outer covering of stethoscope cover 100, thus tearing or cutting through the covering to split it open and facilitate easy removal of stethoscope 200 from stethoscope cover 100 after use. Rip cord 140 is preferably a continuous piece of material running the length of stethoscope cover 100 from pull tab 135 to or through head 110. Rip cord 140 and pull tab 135 can be made out of, but are not limited to, nitrile, natural latex, synthetic latex, blended latex, plastic, nylon, neoprene, thermoplastic elastomers (“TPE”), thermoplastic polyurethane (“TPU”), polyvinyl chloride (“PVC”), chloroprene, polyisoprene, vinyl, rubber, butyl rubber, silicone, string, thread, monofilament nylon, a string, or any combination thereof, and other materials that can be formed into an elongated and flexible string-like or rod-like. Stethoscope cover 100, is most preferably formed from Nitrile, a synthetic rubber copolymer of acrylonitrile and butadiene. Although Nitrile is most preferably used, desirable results can nonetheless be obtained by forming stethoscope cover 100 and its corresponding portions from other synthetic rubbers, natural rubber, silicone or any other desirable material. Stethoscope cover 100 can also be made of any other flexible and stretchable material or composition. In one embodiment, most of stethoscope cover 100 comprises a thickness of about 0.1016 millimeters (“mm”) to about. 1524 mm.

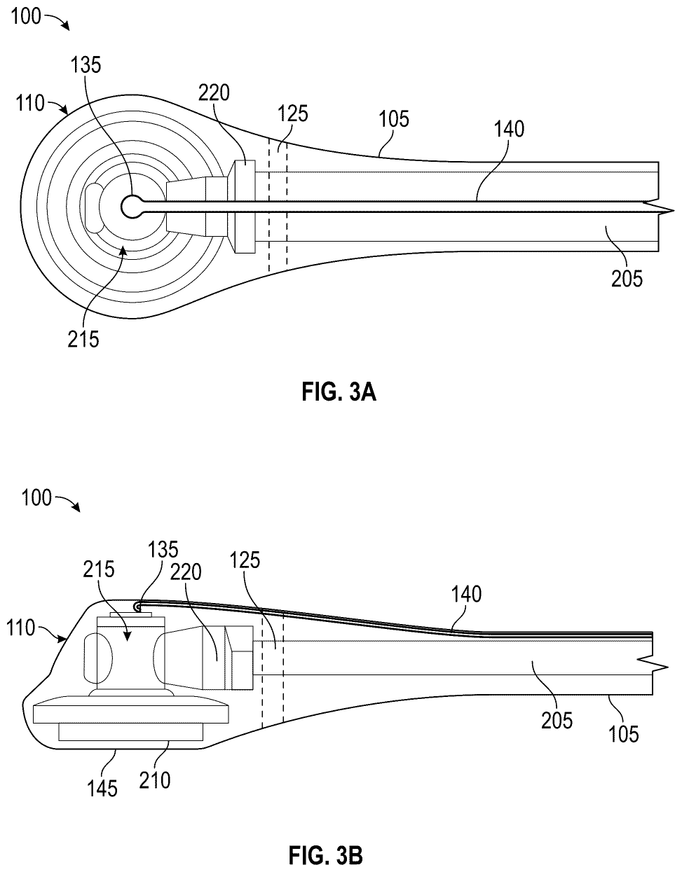

As best illustrated in FIGS. 1-3B, pull tab 135 can extend from at or near lip 120 and rip cord can terminate at or in head 110. In this embodiment, to remove stethoscope 200 from stethoscope cover 100, a user holds an end portion of flared end 130 and draws pull tab 135 toward head 110, thus causing rip cord 140 to tear or otherwise cut through flared end 130, neck 105 and tension band 125 and a portion of head 110. In one embodiment, a terminal end portion of rip cord 140 can be widened (see FIG. 3A) to better secure it to a portion of the stethoscope cover 100 to help prevent rip cord 140 from being pulled out or otherwise removed from the rest of stethoscope cover 100 when it is being used to split stethoscope cover 100 open.

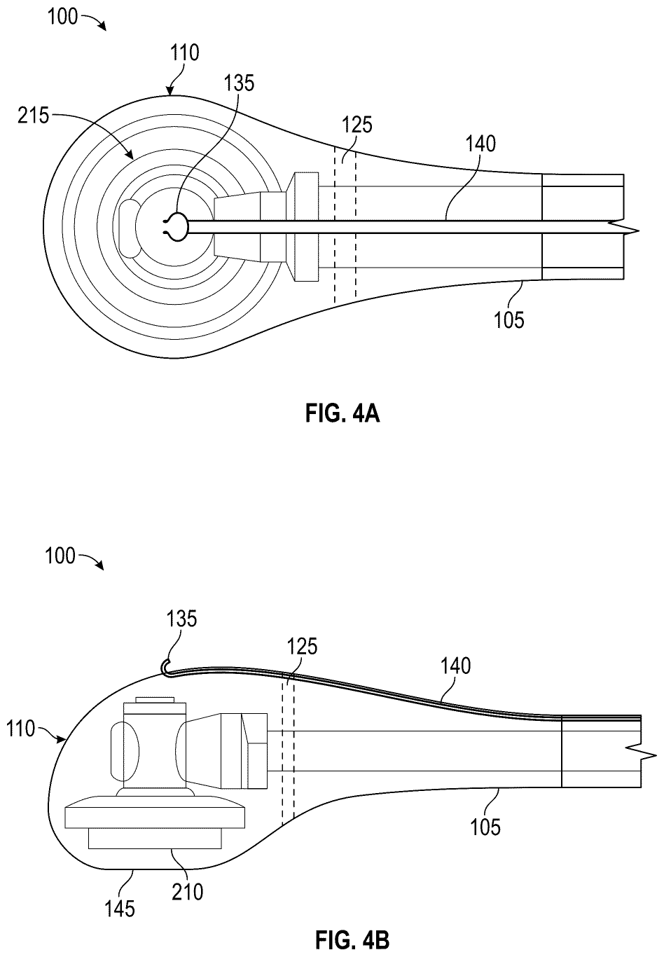

FIGS. 4A and 4B illustrate an embodiment wherein pull tab 135 preferably extends from within head 110 and rip cord 140 extends along the length of head 110, neck 105, flared end 130, and lip 120. In this embodiment, a user holds head 110 while pulling pull tab 135 up toward lip 120 such that rip cord 140 tears through a portion of head 110, then neck 105 and then through lip 120, thus splitting stethoscope cover 100 open along at least a substantial portion of its entire length—for example along at least 80% of its entire length and more preferably along at least 90% of its entire length. In one embodiment, rip cord 140 is preferably able to tear or cut through tension band 125 when splitting stethoscope cover 100. Optionally, rip cord 140 can extend from flared end 130 to (or about to) tension band 125, which that when stethoscope cover 100 is split open by rip cord 140, rip cord 140 does not split open head 110 and instead a user simply removes chest piece 215 of stethoscope from head portion 110.

Optionally, pull tab 135 can extend from both ends of rip cord 140 such that a user can split stethoscope cover 100 by pulling the bottom pull tab 135 upward or by pulling a top pull tab 135 downward.

In one embodiment, rip cord 140 and tension band 125 are embedded within stethoscope cover 100. Stethoscope cover 100, rip cord 140, and tension band 125 are preferably made at the same time, with the same pour or injection or other formation method of material that forms the rest of stethoscope cover 100.

Head 110 can include flat side 145, to accommodate diaphragm 210 (i.e., that portion of diaphragm 210 that comes into contact with a patient while a healthcare worker uses stethoscope 200 within stethoscope cover 100 on a patient). Flat side 145 improves transmission of sound through head 110 and into stethoscope 200. The side of head 110 opposite to flat side 145 can be any desired shape, but in one embodiment is preferably curved or rounded.

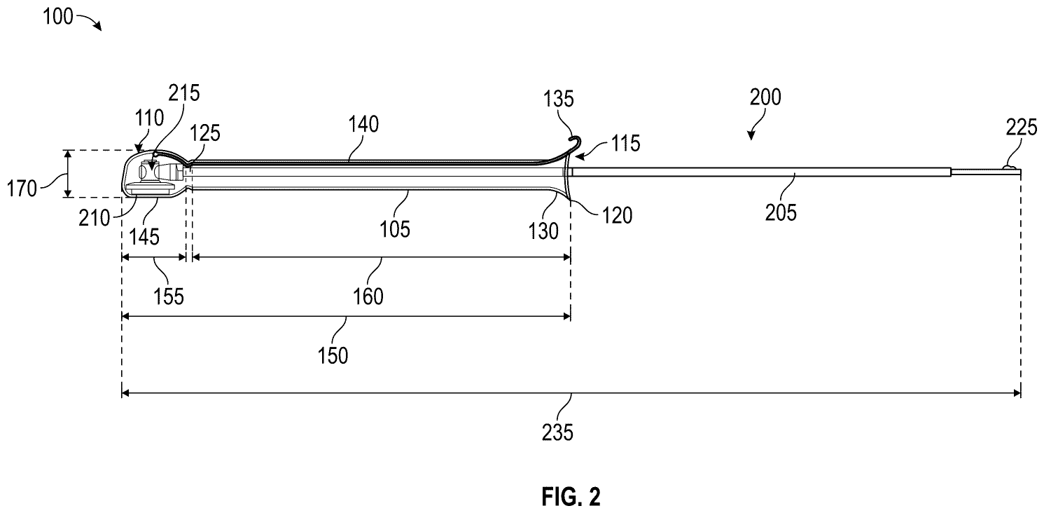

FIG. 2 shows a side view of stethoscope cover 100 covering stethoscope 200. Pull tab 135 of rip cord 140 can be seen extending near lip 120. Rip cord 140 can be placed anywhere along the circumference of stethoscope cover 100 but is preferably placed on the side opposite flat side 145. Preferably, head 110 extends head length 155 of stethoscope cover 100, neck 105 extends neck length 160 stethoscope cover 100. Head length 155 is measured from a terminal end portion of head 110 to tension band 125. Total cover length 150 includes head length 155 and neck length 160. Neck length 160 preferably extends from tension band 125 (if tension band 125 is provided) to an end of stethoscope cover 100 at lip 120. If tension band 125 is not provided, neck length 160 preferably extends from a proximal end of head 110 to an end of stethoscope cover 100 at lip 120. Preferably, total cover length 150 extends anywhere from about one-third to about two-thirds of an overall length of stethoscope length 235—the overall length of stethoscope length 235 being the length from the far end of diaphragm 210 to the opposite far end of earpiece 225.

In one embodiment, head length 155 is less than ⅓ as long as neck length 160. In one embodiment, head length 155 is less than ⅕ as long as neck length 160. In one embodiment, total cover length 150 is preferably at least six inches. In one embodiment, total cover length 150 is preferably at least ten inches.

Stethoscope cover 100 also has head width 165 and head height 170. Head length 155, head width 165, and head height 170 are preferably sized such that diaphragm 210 fits inside head 110 in a way that is tight such that a portion of head portion 110 is in tension when chest piece 215 is disposed therein. Neck 105 has a circumference and length such that at least a length of tubing 205 of stethoscope 200 fits inside neck 105. In one embodiment, a diameter of neck 105 can be selected such that when stethoscope 200 is disposed within stethoscope cover 100 at least a portion of neck 105 is under tension. Flared end 130 leading to lip 120 can be sized and shaped to fit various sizes of stethoscope 200. Optionally, a second tension band can be disposed or otherwise formed in neck 105 of stethoscope cover 100 such that when stethoscope 200 is disposed within stethoscope cover 100, tension of the second tension band, squeezes against one or more of tubes 205 to help prevent flared end 130 from falling down toward head 110.

In one embodiment, stethoscope cover 100 does not have flared end 130 and/or lip 120. In this embodiment, neck 105 extends from head 110 and neck 105 has opening 115. In another embodiment, stethoscope cover 100 does not have flared end 130, but does have lip 120, but now lip 120 is disposed on an end portion of neck 105.

FIGS. 3A and 3B illustrate a closeup of an end portion of stethoscope cover 100, having neck 105 and head 110, which can be positioned over diaphragm 210 of stethoscope 200.

Most preferably, stethoscope cover 100 is shaped and sized such that opening 115 and lip 120 is disposed on or about tubes 205 or other structures leading to chest piece 215 of stethoscope 200. Thus, in one embodiment, the entirety of chest piece 215 of stethoscope 200 is disposed within stethoscope cover 100 when stethoscope cover 100 is disposed in its operating configuration on stethoscope 200.

Most preferably, stethoscope cover 100 also encompasses at least an end portion of tubes 205 that attach to and that extend from chest piece 215 of stethoscope 200. In one embodiment, stethoscope cover 100. In one embodiment, stethoscope cover 100 is preferably sized such that the entirety of chest piece 215 is disposed within stethoscope cover 100 when stethoscope 200 is disposed within stethoscope cover 100. Stethoscope cover 100 is preferably large enough such that the user does not need to change his or her typical grasp configuration that they would otherwise use when using stethoscope 200 when using stethoscope 200 while avoiding touching chest piece 215.

In one embodiment, lip 120 can include a flexible strap, and/or elastic member, and can optionally include a fastener or fastening mechanism, including for example hook-and-loop tape, a pressure-sensitive adhesive, twist closure, combinations thereof and the like to help hold lip 120 against one or more tubes 205. In one embodiment, lip 120 is formed from a single continuous piece of material with neck portion 105 (for example like an end portion of a rubber balloon). In one embodiment, if stethoscope cover 100 were filled with water and sealed at opening 115, stethoscope cover 100 can have a shape such that a top view of stethoscope cover 100 is different than a side view of stethoscope cover 100 (for example as illustrated in FIGS. 4A and 4B). Optionally, if stethoscope cover 100 were filled with water and opening 115 were sealed, stethoscope cover 100 can have a shape such that a top view and a side view thereof are the same as one another.

In one embodiment, lip 120 is optionally not provided. In this embodiment, a user can optionally apply an external closure device and/or can simply hold closed the opening 115 with the user's hand and/or fingers clinched around the one or two tubes 205 of stethoscope 200.

The method can include applying stethoscope cover 100 onto an end portion of stethoscope 200 (for example by passing chest piece 215 of stethoscope 200 through opening 115 and into the opposing end portion of stethoscope cover 100), sliding the end portion of stethoscope cover 100 that contains opening 115 along the one or two tubes 205 that extend from chest piece 215 of stethoscope 200 such that stethoscope cover 100 contains the entirety of chest piece 215 of stethoscope 200 and preferably at least some portion of tubes 205 of stethoscope 200 before then securing or allowing to be secured lip 120 such that opening 115 is at least substantially closed around the one or two tubes 205 of stethoscope 200. The user then preferably uses stethoscope 200 on a first patient. When the user is done performing an assessment or otherwise using stethoscope 200 on the first patient, the user preferably removes stethoscope cover 100 by releasing lip 120 and pulling pull tab 135 and rip cord 140, splitting stethoscope cover 100, or otherwise causing lip 120 to no longer be secured on or about the one or two tubes 205 of stethoscope 200, before pulling stethoscope cover 100 away from and off of stethoscope 200. The user then preferably disposes of stethoscope cover 100 and preferably washes or otherwise treats his or her hands before applying a new stethoscope cover 100 when the user is ready to use stethoscope 200 on a second patient.

In one embodiment, as shown in FIGS. 1 and 2, pull tab 135 can extend from lip 120, where rip cord 140 is embedded within and extends at least substantially the length stethoscope cover 100 to head 110. In another embodiment, as shown in FIGS. 3A, 3B, 4A, and 4B, pull tab 135 can extend from head 110, where rip cord 140 is embedded within and travels at least substantially a total length stethoscope cover 100 to lip 120. In another embodiment, rip cord 140 can extend beyond lip 120 and/or head 110 such that pull tab 135 is simply an end portion of material of rip cord 140. In another embodiment, pull tab 135 can be a different material from rip cord 140, and pull tab 135 can be, but is not limited to, a thicker and/or wider piece of material than rip cord 140 so that a user can easily grab onto pull tab 135.

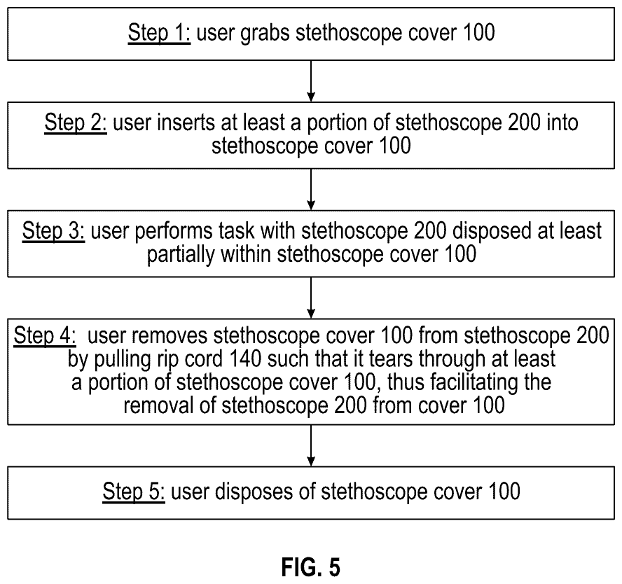

In one embodiment, stethoscope cover 100 can be used to preserve sterility in stethoscope 200. FIG. 5 is a flow diagram showing a method of preserving sterility of stethoscope 200 with stethoscope cover 100. In step 1, a user (a user can be a healthcare worker) grabs stethoscope cover 100 before entering a patient's room. Stethoscope covers 100 can be stored in a receptacle that makes it easy for a healthcare worker to grab one before entering a patient room or other contaminated or potentially contaminated area (similar to how nurses grab disposable gloves before entering a patient's room). In step 2, a user inserts stethoscope 200 into stethoscope cover 100. In one embodiment, stethoscope 200 is preferably sterilized before being inserted into stethoscope cover 100. In step 3: a user performs tasks related to and using stethoscope 200 with stethoscope cover 100 on. In step 4, after completing the tasks, the user removes stethoscope cover 100 from stethoscope 200. Stethoscope cover 100 can be removed by pulling stethoscope cover 100 and sliding it off stethoscope 200. Stethoscope cover 100 can also be removed from stethoscope 200 by pulling pull tab 135 and rip cord 140, splitting stethoscope cover 100, and revealing stethoscope 200 for easy removal of stethoscope cover 100. In step 5, the user disposes of stethoscope cover 100, preserving the sterility of stethoscope 200. In one embodiment, even if stethoscope 200 itself is not sterile, the act of preserving sterility of stethoscope 200 can include only contacting a patient with a sterile covering of stethoscope cover 100.

In one embodiment, head width 165 is approximately 2 inches. In another embodiment, head height 170 is approximately 2 inches. In another embodiment, head length 155 is approximately the total length of diaphragm 210 and stem 220 combined. In another embodiment, total cover length 150, including head length 155 and neck length 160, is approximately less than stethoscope length 235. In another embodiment, neck 105 has a circumference of about ¾ inch. In another embodiment, opening 115 has a diameter of about ½ inch.

The terms, “a”, “an”, “the”, and “said” mean “one or more” unless context explicitly dictates otherwise. Note that in the specification and claims, “about”, “approximately”, and/or “substantially” means within twenty percent (20%) of the amount, value, or condition given.

Embodiments of the present invention can include every combination of features that are disclosed herein independently from each other. Although the invention has been described in detail with particular reference to the disclosed embodiments, other embodiments can achieve the same results. Variations and modifications of the present invention will be obvious to those skilled in the art and this application is intended to cover, in the appended claims, all such modifications and equivalents. The entire disclosures of all references, applications, patents, and publications cited above are hereby incorporated by reference. Unless specifically stated as being “essential” above, none of the various components or the interrelationship thereof are essential to the operation of the invention. Rather, desirable results can be achieved by substituting various components and/or reconfiguring their relationships with one another.

What is claimed is:

1. A stethoscope cover comprising:
an elongated neck portion, said elongated neck portion comprising an opening at a proximal end thereof;
a head disposed at a terminal end portion of said elongated neck portion, said head configured to accommodate a chest piece of the stethoscope; and
a rip cord disposed within or positioned inside of said elongated neck portion.

2. The stethoscope cover of claim 1 further comprising a tension band defining a proximal end of said head.

3. The stethoscope cover of claim 2 wherein said tension band delineates a transition between said head and said elongated neck portion.

4. The stethoscope cover of claim 2 wherein said tension band comprises a thickness that is greater than a thickness of said head.

5. The stethoscope cover of claim 1 wherein said rip cord extends at least from said opening to said head.

6. The stethoscope cover of claim 5 wherein said rip cord extends at least from said opening and terminates within said head.

7. The stethoscope cover of claim 1 wherein said rip cord comprises a pull tab extending beyond said opening of said elongated neck portion.

8. The stethoscope cover of claim 1 wherein said rip cord is embedded within material that at least forms said elongated neck portion.

9. The stethoscope cover of claim 1 wherein said rip cord is adhered to an internal surface of at least said elongated neck portion.

10. The stethoscope cover of claim 1 wherein said rip cord comprises a pull tab extending from said head of said stethoscope.

11. The stethoscope cover of claim 1 wherein said head comprises a flat portion that is positioned to accommodate a diaphragm of a chest piece of a stethoscope when a stethoscope is disposed within said stethoscope cover and wherein said head comprises dimensions that accommodate a chest piece of the stethoscope.

12. The stethoscope cover of claim 11 wherein when said rip cord is positioned opposite from said flat portion.

13. The stethoscope cover of claim 1 wherein said stethoscope cover further comprises a lip formed around said opening.

14. The stethoscope cover of claim 1 wherein said proximal end of said elongated neck portion is flared such that said opening comprises a diameter that is larger than a diameter of a rest of said elongated neck portion.

15. A method of preserving sterility of a stethoscope, the method comprising:
disposing a stethoscope at least partially within a cover such that a chest piece of the stethoscope is positioned within a head of the cover and such that at least a portion of tubing of the stethoscope is disposed within a neck portion of the cover;
pressing the head of the cover, having the chest piece disposed therein, against a patient to perform an auscultation;
after the auscultation, pulling a rip cord such that the rip cord tears or otherwise cuts through at least a portion of the cover to facilitate removal of at least a portion of the stethoscope from the cover; and
removing the cover from the stethoscope.

16. The method of claim 15 wherein pulling a rip cord comprises a user grasping a pull tab and pulling the pull tab to pull the rip cord.

17. The method of claim 15 further comprising disposing of the cover after the removing step.

18. The method of claim 17 further comprising disposing the stethoscope at least partially within a new cover after the disposing step.

19. The method of claim 15 wherein pulling a rip cord comprises pulling the rip cord from a top opening of the cover toward the head.

20. The method of claim 15 wherein pulling a rip cord comprises pulling the rip cord from the head toward a top opening of the cover.
